(19) United States
(12) Patent Application Publication
(10) Pub. No.: US 2005/0287205 A1
(43) Pub. Date: Dec. 29, 2005

(54) Dietary supplement compositions

(76) Inventors: Meiling Wang, East Brunswick, NJ (US); Zhuying Wang, Dayton, NJ (US); Fangliang Zhang, Fanwood, NJ (US); Luquan Wang, East Brunswick, NJ (US)

Correspondence Address: FOX ROTHSCHILD O'BRIEN & FRANKEL LLP;PRINCETON PIKE CORPORATE CENTER, 997 LENOX DRIVE, BUILDING 3, LAWRENCEVILLE, NJ, 08648, US

(21) Appl. No.: 11/158,433

(22) Filed: Jun. 22, 2005

(30) Foreign Application Priority Data
Jun. 22, 2004 (CN) 200410024282.5

(51) Int. Cl. A61K 31/724; A61K 31/716; A61K 9/48; A61K 9/20; A61K 31/19

(52) U.S. Cl. 424/451; 424/464; 514/557; 514/574; 514/54; 514/57; 514/58

(57) ABSTRACT

A dietary supplement composition, dosage forms, and methods of use are provided which comprise an effective amount of at least one compound selected from the group consisting of acetic acid, citric acid, and malic acid; and, at least one carrier selected from the group consisting of a cyclodextrin, a porous starch, a KONJAC powder, and a carboxyl methyl cellulose (CMC).

Priority is derived herein from Peoples Republic of China National Application No. 200410024282.5, filed on Jun. 22, 2004.

### FIELD OF THE INVENTION

The invention generally relates to dietary supplement compositions which comprise an effective amount of at least one food acid selected from the group consisting of acetic acid (e.g., vinegar), citric acid, and malic acid; and, at least one diluent, excipient or carrier selected from the group consisting of cyclodextrin, porous starch, KONJAC powder, and carboxyl methyl cellulose (CMC). Vinegar, particularly apple cider vinegar, is a preferred element of compositions of the present invention. Vinegar, for example, provides acetic acid, inter alia, in the compositions for the control of blood pressure and triglyceride, stimulating metabolism. The invention also relates to methods of manufacturing tablets or capsules containing relatively high concentrations of vinegar, e.g., apple cider vinegar.

### BACKGROUND OF THE INVENTION

A need continues to exist for an inexpensive food supplement based on simple and inexpensive ingredients to provide substantial health benefits. Vinegar, for example, is used traditionally as a folk medicine and is believed to have several beneficial effects such as improving appetite, enhancing mineral absorption and speeding recovery from fatigue. One of the major components of vinegar is acetic acid. However, vinegar, for example, is particularly difficult to ‘administer’ orally, e.g., in a relatively large amount with food, because of its very strong taste. Although, apple cider vinegar tablets, for example, are commercially available, the concentration of acetic acid in previous and currently available compositions are ineffectively low and do not address the biological need and/or exhibit the efficacy of compositions described herein.

### SUMMARY OF THE INVENTION

Dietary supplement compositions are described which comprise an effective amount of at least one compound selected from the group consisting of acetic acid, citric acid, and malic acid; and, at least one carrier selected from the group consisting of a cyclodextrin, a porous starch, a KONJAC powder, and a carboxyl methyl cellulose (CMC); wherein, upon oral administration, provide treatment and confer improvement in human conditions such as controlling high blood pressure, controlling high triglyceride level, mediating weight loss, stimulating calcium absorption, providing relief and/or recovery from fatigue, and other related biological conditions.

A further object of the instant invention is to provide methods for making such dietary supplement which contain high concentration, i.e., therapeutically effective amounts, of acetic acid, for example.

### DETAILED DESCRIPTION OF THE INVENTION

The present invention is directed to an inexpensive, easily produced yet efficacious dietary supplement for the relief of common human medical problems such as over-weight, high blood pressure and high level of triglyceride, poor digestion, and metabolism disorders.

Orally administered acetic acid is immediately absorbed. Subsequent uptake occurs in the liver and peripheral tissues. Acetic acid is metabolized via acetyl-CoA in the tricarboxylic acid cycle in liver and skeletal muscle. Acetic acid administration accordingly greatly stimulates the tricarboxylic acid (TCA) cycle and metabolism. The resulting enhanced burning of energy contributes significantly to weight loss and, for example, as a corollary, reduced triglyceride level. Takashi, et al., moreover, have demonstrated that acetic acid administration to mammals enhances glycogen repletion in liver and skeletal muscle thus to significantly promote recovery from fatigue. Acetic acid, per se, a significant element of vinegar, as further discussed herein, furthermore significantly reduces both blood pressure and renin activity. See, e.g., Kondo S, et al., Antihypertensive effects of acetic acid and vinegar on spontaneously hypertensive rats. Biosci Biotechnol Biochem. 2001 Dec;65(12):2690-2694. 1.6 mL vinegar/100 g diet, for example, significantly enhances the intestinal absorption of calcium, for example. Kishi M,., Enhancing effect of dietary vinegar on the intestinal absorption of calcium in ovariectomized rats. Biosci Biotechnol Biochem. 1999 May;63(5):905-10.

Acetic acid is an example preferred component of dietary supplement compositions described herein. The acetic acid in compositions for oral administration described herein is absorbed, uptaken, and metabolized via acetyl-CoA in the TCA cycle. Acetic acid greatly stimulates the TCA cycle and metabolism. The enhanced oxidation, burning of energy, mediates weight loss and reduced triglyceride level, for example.

When pure, acetic acid is a clear, colorless liquid with a sharp, irritating odor of vinegar. In poorly heated laboratories, the acid was oftentimes found frozen inside its container because its freezing point is only slightly below room temperature at 16.7° C. The term glacial (ice-like) came to be applied to the pure acid in either its solid or liquid state. Glacial acetic acid boils at 118° C., and has a density of 1.049 g/mL at 25° C. It is flammable with a flash point of 39° C. Through hydrogen-bonding interactions, acetic acid is miscible (mixable) in all proportions with water, ethyl alcohol, and diethyl ether.

Vinegar generally contains 4-8% acetic acid by volume. Vinegar also contains valuable components generally including, an effective amino acid population. Production of vinegar is well known in the art. Apple cider vinegar, for example, is commercially available in large quantities. Vinegar, however, can be produced by fermenting a myriad of substances, including but not limited to apple cider, fruit material, grains, solutions of starch, sugar solutions, coconut water or alcoholic foodstuffs such as wine.

Citric acid is versatile, widely used, inexpensive, and safe food addictive. Citric acid is widely respected for relieving conditions of fatigue, poor digestion, cold and flu infections, asthma, hypertension and cholesterol deposits in blood vessels. It is an important metabolite in virtually all living organisms and is especially abundant naturally in citrus fruits and berries. It is another example effective component of dietary supplemental compositions described herein. Citric acid is also metabolized in the tricarboxylic acid cycle. Oral administration of citric acid also greatly stimulates the tricarboxylic acid cycle and metabolism.

Malic acid, an alpha-hydroxy organic acid found in apples and other fruits, is sometimes referred to as a fruit acid. Malic acid, in the form of its anion, malate, is a key intermediate in the citric acid cycle (Krebs cycle), therefore feeding malic acid will stimulate the tricarboxylic acid cycle and metabolism. Malic acid aids in exercise recovery by counteracting the buildup of lactic acid due to the consumption of lactic acid in active tricarboxylic acid cycle and metabolism. Malic acid is yet another example effective component of dietary supplement compositions of the present invention.

Dietary supplements described herein reduce pain in the joints of knees and shoulders. The two primary sources of chronic back pain are muscular hypertonicity resulting in joint compression and possible nerve impingement, and lactic acid buildup in hypertonic muscles creating nociceptor irritation. Improper or insufficient movement, trauma, and/or postural habits lead to chronic muscular hypertonicity and chronic muscular hypertonicity contributes to lactic acid build-up in muscle and thus, to back pain. Therefore, whether muscular hypertonicity results from pain or produces it, whether muscular hypertonicity arises from physical or emotional origin, the result is the same: lactic acid build-up. Lacic acid (in the form of its salts, called lactates) can be either converted to pyruvic acid, which then enters the tricarboxylic acid cycle to produce energy or converted into glucose or glycogen which enters glycolysis to produce energy. Acetic acid administration greatly stimulates the tricarboxylic acid (TCA) cycle and therefore speeds up the conversion of lactate (from lactic acid) to pyruvic acid, thus to reduce lactic acid build-up and reduce pain.

Cyclodextrins are bucket-shaped oligosaccharides well-known in the art that are generally produced from starch. Their molecular structure confers a unique ability to act as molecular carriers of active ingredients of compositions of the present invention. Here, cyclodextrins, and derivatives thereof, are preferred components of compositions described herein. Cyclodextrins generally mask the taste and odor of vinegar, for example, and also to reduce dermal, gastrointestinal irratation.

Many different chemical moieties may be introduced into the Cyclodextrin molecule by reaction with the hydroxyl groups lining the upper and lower ridges of the toroid; for example, hydroxypropyl, carboxymethyl, and acetyl. Since each Cyclodextrin hydroxyl groups differs in its chemical reactivity, reaction processes produces an amorphous mixture of thousands of positional and optical isomers. Preferred examples of chemically modified cyclodextrins as components of formulations of the present invention include, but are not limited to, 2-hydroxypropyl-beta-Cyclodextrin, 2-hydroxypropyl-gamma-Cyclodextrin, and hydroxyethyl-beta-Cyclodextrin. Cyclodextrin molecules (alpha, beta, or gamma) can have up to 3(n) substituents, where n is the number of glucopyranose units of the Cyclodextrin molecule. This is referred to as the degree of substitution (DS). The DS refers to substituents other than hydrogen; substituents may be all of one kind or mixed. Non-integer degrees of substitution occur as weighted averages are used to describe substitutional variability. See, e.g., Volume 3 (cyclodextrins) of the 11 Volume Collection “Comprehensive Supramolecular Chemistry”, available through Elsevier Science Inc., 660 White Plains road, Tarrytown, N.Y., 10591-5153 USA. See, also, Pitha, Josef, U.S. Pat. No. 4,727,064, Pharmaceutical Preparations Containing Cyclodextrin Derivatives; Muller, B. W., U.S. Pat. No. 4,764,604, Derivatives of Gamma Cyclodextrins; Yoshida, A., et al., (1988) Int. Pharm, Vol. 46, p. 217: Pharmaceutical Evaluation Of Hydroxy Alkyl Ethers Of B-Cyclodextrins; Muller, B. W., (1986). J. Pharm Sci. 75, No 6, Jun. 1986: Hydroxypropyl-B-Cyclodextrin Derivatives: Influence Of Average Degree Of Substitution On Complexing Ability And Surface Activity; Irie, T., et al., (1988) Pharm Res., No 11, p. 713: Amorphous Water-Soluble Cyclodextrin Derivatives: 2-hydroxyethyl, 3-hydroxypropyl, 2-hyroxyisobutyl, and carboxamidomethyl derivatives of B-cyclodextrin.

Porous starch is a kind of functional starch with special hollow structure, which makes porous starch capable to adsorb a variety of materials, i. e. liquids such as water, oils, and ethanol. It is employed in example compositions of the present invention as a carrier for liquid acetic acid or vinegar. See, e.g., J Biomater Sci Polym Ed. 2001;12(11):1227-41; Starch-based biodegradable hydrogels with potential biomedical applications as drug delivery systems, Biomaterials. 2002 May;23(9): 1955-66.

Carboxyl methyl cellulose (CMC), as used herein refers to a water-soluble derivations of cellulose. CMC and its derivatives, referred to herein generally as “Carboxyl methyl cellulose” or CMC, are used as a thickener, and also a binder of acetic acid or vinegar in compositions of the present invention. Hydroxypropyl methylcellulose (HPMC), for example, and related excipients, including but not limited to polyvinylpyrrolidone, polydextrose, and polyvinylalcohol, for example, as known in the art of pharmaceutical formulation, may also be employed as carriers, for example, in compositions and controlled-release formulations of the present invention.

An aqueous product is dried, for example, in a production process of compositions of the present invention. Acetic acid, for example, and/or other active ingredient(s) described herein, at this time in the process, are absorbed to either porous starch and/or the cyclodextrin and/or carboxyl methyl cellulose (CMC) elements of the composition.

KONJAC powder, another example component of compositions described herein, contain a significant quantity of polysaccharide, glucomannan, as dietary fiber with special properties such as gelatinizing agent, intestine cleaning agent, cholesterol and blood sugar reducing agent. The term “KONJAC powder” as used herein also refers to powderized glucomannan and derivatives of glucomannan. Konjac Foods is an example commercial source. 355 W. Olive Ave., Suite 104, Sunnyvale, Calif. 94086.

The general weight percentage range for compounds selected from the group consisting of acetic acid, citric acid, and malic acid is 5-99%. The preferred weight percentage range for compounds selected from the group consisting of acetic acid, citric acid, and malic acid is 20-75%. The most-preferred weight percentage range for compounds selected from the group consisting of acetic acid, citric acid, and malic acid is 30-50%.

The general weight percentage range for carriers selected from the group consisting of a cyclodextrin, a porous starch, a KONJAC powder, and a carboxyl methyl cellulose (CMC) is 1-40%. The preferred weight percentage range for carriers selected from the group consisting of a cyclodextrin, a porous starch, a KONJAC powder, and a carboxyl methyl cellulose (CMC) is 4-25%. The most-preferred weight percentage range for carriers selected from the group consisting of a cyclodextrin, a porous starch, a KONJAC powder, and a carboxyl methyl cellulose (CMC) is 4-16%. For example, the carrier includes 1-4% of cyclodextrin, 1-5% of porous starch, 1-6% of KONJAC powder, and 1-6% of food-grade carboxyl methyl cellulose (CMC).

The resulting mixture of compounds selected from the group consisting of acetic acid, citric acid, and malic acid and the carrier selected from the group consisting of a cyclodextrin, a porous starch, a KONJAC powder, and a carboxyl methyl cellulose (CMC) is mixed thoroughly, and is baked for 2-3 hours at about 40±5° C. The resulting dried material is then 80-100 mesh powderized and compressed into tablets or used to fill capsules as is well-known in the art.

Cummings J. H., et al., Short chain fatty acids in human large intestine, portal, hepatic and venous blood. Gut 1987;28:1221-1227.

Pomare E. W., et al., Carbohydrate fermentation in the human colon and its relation to acetate concentrations in venous blood. J. Clin. Investig. 1985;75:1448-1454.

Ballard F. J., Supply and utilization of acetate in mammals. Am. J. Clin. Nutr. 1972;25:773-779.

Crabtree B., Gordon M., Christie S. L. Measurement of the rates of acetyl-CoA hydrolysis and synthesis from acetate in rat hepatocytes and the role of these fluxes in substrate cycling. Biochem. J. 1990;270:219-225.

Spydevold O., Davis E. J., Bremer J. Replenishment and depletion of citric acid cycle intermediates in skeletal muscle. Eur. J. Biochem. 1976;71:155-165.

Takashi Fushimi, et al., Acetic Acid Feeding Enhances Glycogen Repletion in Liver and Skeletal Muscle of Rats. Journal of Nutrition. 2001;131:1973-1977.

Kondo S, et al., Antihypertensive effects of acetic acid and vinegar on spontaneously hypertensive rats. Biosci Biotechnol Biochem. 2001 Dec;65(12):2690-2694.

Kishi M,., Enhancing effect of dietary vinegar on the intestinal absorption of calcium in ovariectomized rats. Biosci Biotechnol Biochem. 1999 May;63(5):905-10

### EXAMPLES

### Example I

Measured by weight percentage, 25% of food-grade glacial acetic acid, 60% of apple cider vinegar, 2% parts of cyclodextrin, 2% parts of porous starch, 2% of KONJAC powder, and 2% of food-grade carboxyl methyl cellulose (CMC) are mixed thoroughly. The resulting mixture is baked for 2 hours at 40° C. The dried material is then powderized by means of 100 mesh and used for making tablets or capsules using procedures that are recognized in the art.

### Example II

Measured by weight percentage, 30% of food-grade glacial acetic acid, 59% of apple cider vinegar, 2% of cyclodextrin, 3% of porous starch, 3% of KONJAC powder, and 3% of food-grade carboxyl methyl cellulose (CMC) are mixed thoroughly. The resulting mixture is baked for 2 hours at 45° C. The dried material is then powderized by means of 90 mesh and used for making tablets or capsules using procedures that are recognized in the art.

### Example III

Measured by weight percentage, 20% of food-grade glacial acetic acid, 76% of apple cider vinegar, 1% of cyclodextrin, 1% of porous starch, 1% of KONJAC powder, and 1% of food-grade carboxyl methyl cellulose (CMC) are mixed thoroughly. The resulting mixture is baked for 3 hours at 38° C. The dried material is then powderized by means of 100 mesh and used for making tablets or capsules using procedures that are recognized in the art.

### Example IV

Measured by weight percentage, 10% of food-grade glacial acetic acid, 86% of apple cider vinegar, 1% of cyclodextrin, 1% of porous starch, 1% of KONJAC powder, and 1% of food-grade carboxyl methyl cellulose (CMC) are mixed thoroughly. The resulting mixture is baked for 2 hours at 38° C. The dried material is then powderized by means of 100 mesh and used for making tablets or capsules using procedures that are recognized in the art.

### Example V

Measured by weight percentage, 50% of food-grade glacial acetic acid, 29% apple cider vinegar, 4% of cyclodextrin, 5% of porous starch, 6% of KONJAC powder, and 6% of food-grade carboxyl methyl cellulose (CMC) are mixed thoroughly. The resulting mixture is baked for 2 hours at 40° C. The dried material is then powderized by means of 100 mesh and used for making tablets or capsules using procedures that are recognized in the art.

### Example VI

Measured by weight percentage, 20% of food-grade glacial acetic acid, 73% of apple cider vinegar, 1% of cyclodextrin, 2% of porous starch, 2% of KONJAC powder, and 2% of food-grade carboxyl methyl cellulose (CMC) are mixed thoroughly. The resulting mixture is baked for 3 hours at 38° C. The dried material is then powderized by means of 100 mesh and used for making tablets or capsules using procedures that are recognized in the art.

It is to be understood that the present invention is not limited to embodiments described above, but encompasses any and all embodiments within the scope of the following claims.

1. A dietary supplement composition comprising an effective amount of at least one compound selected from the group consisting of acetic acid, citric acid, and malic acid; and, at least one carrier selected from the group consisting of a cyclodextrin, a porous starch, a KONJAC powder, and a carboxyl methyl cellulose (CMC).

2. A composition according to claim 1 comprising at least two carriers selected from the group consisting of cyclodextrin, porous starch, KONJAC powder, and carboxyl methyl cellulose (CMC).

3. A composition according to claim 2 comprising at least three carriers selected from the group consisting of cyclodextrin, porous starch, KONJAC powder, and carboxyl methyl cellulose (CMC).

4. A composition according to claim 1 which comprises a cyclodextrin carrier.

5. A composition according to claim 1 which comprises a porous starch carrier.

6. A composition according to claim 1 which comprises a KONJAC powder carrier.

7. A composition according to claim 1 which comprises a carboxyl methyl cellulose (CMC) carrier.

8. A composition according to claim 1 which comprises an effective amount of acetic acid and residual components derived from vinegar.

9. A composition according to claim 1 which comprises an effective amount of at least two compounds selected from the group consisting of acetic acid, citric acid, and malic acid.

10. A composition according to claim 9 which comprises an effective amount of acetic acid and residual components derived from vinegar.

11. An oral dosage form selected from the group consisting of a tablet and a capsule which comprises a composition comprising an effective amount of at least one compound selected from the group consisting of acetic acid, citric acid, and malic acid; and, at least one carrier selected from the group consisting of a cyclodextrin, a porous starch, a KONJAC powder, and a carboxyl methyl cellulose (CMC).

12. An oral dosage form according to claim 11 which comprises at least two carriers selected from the group consisting of cyclodextrin, porous starch, KONJAC powder, and carboxyl methyl cellulose (CMC).

13. An oral dosage form according to claim 11 which comprises an effective amount of acetic acid and residual components derived from vinegar.

14. An oral dosage form according to claim 13 which comprises an effective amount of at least two compounds selected from the group consisting of acetic acid, citric acid, and malic acid.

15. A method of treatment of a person comprising orally administering a composition comprising an effective amount of at least one compound selected from the group consisting of acetic acid, citric acid, and malic acid; and, at least one carrier selected from the group consisting of a cyclodextrin, a porous starch, a KONJAC powder, and a carboxyl methyl cellulose (CMC).

16. A method of treatment according to claim 15 for the control of blood glucose level.

17. A method of treatment according to claim 15 for the control of blood pressure.

18. A method of treatment according to claim 15 for the control of blood cholesterol level.

19. A method of treatment according to claim 15 for the control of blood triglyceride level.

20. A method of treatment according to claim 15 for the control of body weight and/or for the reduction of pain in the joints of knees and shoulders.
